(19) United States
(12) Patent Application Publication
(10) Pub. No.: US 2026/0295079 A1
(43) Pub. Date: Oct. 1, 2026

(54) MODIFIED AAV VECTORS FOR GENE THERAPY

(71) Applicant: THE TRUSTEES OF INDIANA UNIVERSITY, Bloomington, IN (US)

(72) Inventors: Weidong XIAO, Dresher, PA (US); Anh Kim LAM, Indianapolis, IN (US)

(21) Appl. No.: 19/100,720

(22) PCT Filed: Aug. 24, 2023
(86) PCT No.: PCT/US2023/072826

§ 371 Date: Feb. 3, 2025

Related U.S. Application Data
(60) Provisional application No. 63/400,880, filed on Aug. 25, 2022.

(51) Int. Cl. A61K 48/00; A61K 47/54; A61K 47/69; C07K 14/005; C12N 15/86

(52) U.S. Cl. CPC A61K 48/0041; A61K 47/545; A61K 47/6901; C07K 14/005; C12N 15/86; C12N 2750/14122; C12N 2750/14143; C12N 2750/14145

(57) ABSTRACT

Disclosed herein are compositions comprising chemically modified adeno-associated viruses (AAV) engineered by chemical modification of capsid proteins, whether wildtype or engineered, and methods of using such modified AAVs for therapeutic use wherein the modified AAVs are re-directed to specific organs/tissues relative to the unmodified AAV.

### CROSS-REFERENCE TO RELATED APPLICATIONS

This application claims priority to U.S. Provisional Application Ser. No. 63/400,880, filed Aug. 25, 2022, the entire contents of which are incorporated herein by reference.

### GOVERNMENT LICENSE RIGHTS

This invention was made with government support under HL130871 and HL142019 awarded by National Institutes of Health. The Government has certain rights in the invention.

### BACKGROUND

Gene therapy is the delivery of exogenous nucleic acids to correct or replace defective genes. Among viral vectors available for gene therapy, the adeno-associated virus (AAV) has become the most popular modality in biotechnology today. However, despite the approved AAVs following clinical trials, challenges and limitations remain. For instance, current protocols require administering a high dose of AAV, and many human subjects have developed pre-existing neutralizing antibodies against many AAV serotypes. Finally, therapeutic AAVs have broad bio-distribution when administered to a subject. Accordingly, an unmet need exists for modified AAVs capable of preferential uptake of a single strand of DNA to one or more specific cell type.

### SUMMARY

Disclosed herein are modified AAV with chemical moieties that alter the tropism of the original AAV. In this regard, a modified AAV may be used as a delivery vehicle for recombinant DNA, or other biomolecules, to specific cell types rather than a broad bio-distribution. With the use of targeted delivery of infection, less virus could be administered to a subject and less impact on a subject's organs.

In accordance with one embodiment of the present disclosure modified AAVs are provided that have been produced using chemical modification of the capsids that can re-direct the modified AAV to specific organs/tissues for therapeutic use. More particularly, in one embodiment the modified disclosed herein significantly improve the tropism of the vectors towards muscle and the bone marrow tissues of a subject.

In accordance with one embodiment a modified AAV includes a single stranded recombinant DNA is provided comprising capsid protein, wherein the capsid protein has been modified by the covalent linkage of a non-native chemical moiety to the capsid protein. This modified AAV with recombinant DNA may be referred to as a modified rAAV. More particularly, the covalent linkage of the chemical moiety to the capsid protein alters the tropism of the modified rAAV relative to the original unmodified rAAV. In accordance with on one embodiment the modified rAAV is selected from wild-type serotypes such as: AAV2, AAV5x, AAV8, and AAV9, and the chemical moiety is selected from the group consisting of 5-(Dimethylamino) naphthalene-1-sulfonyl chloride (Dc) and N-ethylmaleimide (NEM). In one embodiment the chemical moiety is 5-(Dimethylamino) naphthalene-1-sulfonyl chloride (Dc), and the altered tropism is an enhanced preferential uptake of the modified AAV, relative to the original unmodified AAV, by muscle tissue. In one embodiment the chemical moiety is N-ethylmaleimide (NEM) and the altered tropism is an enhanced preferential uptake of the chemical modified AAV, relative to the original unmodified AAV, by bone tissue.

In other aspects of the present disclosure, methods are provided for administering a therapeutically effective amount of a modified rAAV described herein for treating a disease or medical condition in a patient. In one embodiment the modified rAAV comprises one or more nucleic acid constructs that encode gene products that treat a disease state. In one embodiment a modified recombinant adeno-associated virus of the present disclosure serves as a delivery vehicle for gene therapy components.

In one embodiment a method of altering the tropism of an AAV is provided, wherein the method comprises chemically modifying a protein of the AAV capsid by the covalent attachment of a chemical moiety to the capsid protein. In one embodiment the AAV to be modified is an rAAV, optionally wherein the rAAV is an rAAV selected from serotypes: AAV2, AAV5x, AAV8, and AAV9. In one embodiment the capsid protein of the rAAV is chemically reacted with the compound 5-(Dimethylamino) naphthalene-1-sulfonyl chloride (Dc; FIG. 1A), N-ethylmaleimide (NEM; FIG. 1B). Advantageously the chemical structure of Dc and NEM bear the functional group of sulfonyl chloride (Dc) or maleimide (NEM), respectively which can be used to covalently link Dc and NEM to an rAAV capsid via an amino group of the capsid as shown in FIGS. 2A and 2B. More particularly, in one embodiment the capsid protein is covalently linked to the chemical moiety 5-(Dimethylamino) naphthalene-1-sulfonyl chloride (Dc) and the altered tropism is an enhanced preferential uptake of the chemical modified AAV, relative to the original unmodified AAV, by muscle tissue. Alternatively, in one embodiment the chemical moiety is N-ethylmaleimide (NEM) and the altered tropism is an enhanced preferential uptake of the chemical modified AAV, relative to the original unmodified AAV, by bone tissue.

In some embodiments, the AAV or rAAV includes an engineered capsid protein. In this manner, the capsid genes vp1, vp2, and/or vp3 are genetically modified resulting in an altered capsid proteins VP1, VP2, and/or VP3. For example AAV5× is an example of an engineered capsid protein. In some embodiments, the AAV comprises an engineered capsid, which is then chemically modified as described herein. In some embodiments, the modified rAAV comprises an AAV including an engineered capsid. In yet another embodiment the AAV to be modified is an rAAV, optionally wherein the rAAV is an rAAV selected from serotypes with engineered capsids. For the purposes of this disclosure capsid proteins and engineered capsid proteins conserve the at least one exposed lysine residue found in VP1, VP2, and/or VP3.

Also encompassed by the present disclosure are pharmaceutical compositions comprising modified recombinant adeno-associated virus described herein and a pharmaceutically acceptable carrier. In accordance with one embodiment a pharmaceutical composition is provided comprising any of the modified recombinant adeno-associated virus described herein in aqueous solutions that are sterilized and optionally stored within various package containers. In other embodiments the pharmaceutical compositions comprise a lyophilized powder. The pharmaceutical compositions can be further packaged as part of a kit that includes a disposable device for administering the composition to a patient. The containers or kits may be labeled for storage at ambient room temperature or at refrigerated temperature.

### BRIEF DESCRIPTION OF THE DRAWINGS

FIG. 1A and FIG. 1B show chemical structure of compounds 5-(Dimethylamino) naphthalene-1-sulfonyl chloride (Dc; FIG. 1A) bearing the functional group of sulfonyl chloride, and and N-ethylmaleimide (NEM; FIG. 1B) which bears the functional group of maleimide.

FIG. 2A and FIG. 2B show AAV vector capsid chemically reacts with sulfonyl chloride compound (FIG. 2A) or maleimide compound (FIG. 2B) to form new engineered capsids; AAV-Dc=recombinant AAV capsid modified with Dc; AAV-NEM=recombinant AAV capsid modified with NEM.

FIG. 3 shows data from an in vitro transduction assays of WT-AAV and modified AAV-Dc on GM16095 cells or AAV-NEM vs. their counterpart WT capsids on HUVEC cells, showing phenotypic change with infectivity of capsid-modified AAVs. Of note, AAV9-NEM significantly enhanced the transduction in HUVEC cells compared to its counter-part unmodified capsid AAV9.

FIG. 4 shows the altered tropism of modified AAV5 with Dc relative to unmodified AAV5 by analyzing qPCR analyses of barcoded WT-AAV (AAV-CB-gluc) and capsid-modified AAV5-Dc was significantly enhanced/redirected to the skeletal hamstring muscle of IV-injected Balb/c mice at week 4 post-injection.

FIG. 5 shows the altered tropism of modified AAV9 with Dc relative to the unmodified AAV9 qPCR analyses of barcoded WT-AAV (AAV-CB-gluc) and capsid-modified AAV9-Dc was significantly enhanced/redirected to the skeletal hamstring muscle of IV-injected Balb/c mice at week 4 post-injection.

FIG. 6 shows qPCR analyses of WT-AAV5 (AAV-CB-eGFP) and capsid-modified AAV5-Dc. Tissue tropism of AAV5-Dc was significantly enhanced/redirected to the hamstring muscle of IV-injected Balb/c mice at week 4.

FIG. 7 shows qPCR analyses of the AAV genome in DNA extracted from mouse tissues IV-injected with AAV9 or AAV9-NEM which was capsid-modified with NEM. Tissue tropism of AAV9-NEM was significantly enhanced/redirected to the bone marrow at week 4.

FIG. 8 shows intact LC-MS of AAV9 (left) and AAV9-Dc (right) deconvoluted spectra show the VP3 protein exact mass (plus VP2 and VP1 also detected) and relative abundance percent of each peak. Chemically engineered capsid AAV9-Dc was characterized with the exact mass of one Dc molecule added to the VP3 protein (See FIG. 9 for a zoomed in spectrum).

FIG. 9 shows a comparison of LC-MS spectra of AAV9 and engineered-capsid AAV9-Dc. Deconvoluted mass spectra of capsid of AAV9 and capsid AAV9-Dc confirm the exact masses of serotype-9 VP proteins and the chemical-engineered capsid 9-Dc's, with the mass gain of 233.2 Da per one Dc molecule conjugation.

FIG. 10 is a screenshots of Agilent MassHunter BioConfirm 8 shows peptide map and sequence coverage of the AAV9 VP3 capsid protein (top: trypsin digested; bottom: chymotrypsin digested).

FIG. 11 shows a MS/MS spectrum of product ions of an AAV9 VP3 peptide sequence.

FIG. 12 shows a LC-MS/MS spectra of the digested capsid proteins of AAV9 (left) vs AAV9-Dc (right). Peptide maps were elucidated with 100% VP3 sequence coverage for both serotypes. The amino acid modified with one Dc molecule was identified as Lys (K) at VP3#326 (See FIG. 10), and the peptide sequence containing this position is shown here for both AAV9 (wild-type) and AAV9-Dc (one Dc-molecule conjugation with the exact mass gain of 233.1 Da detected with one chlorine atom and one hydrogen atom not present in the final product and shown as molecular weight loss).

FIG. 13 shows a spectra comparing wild-type AAV9 and modified AAV9 with NEM.

FIG. 14 shows the bio-distribution of AAV9-NEM relative to AAV9 at week-1 post injection and week-4 post injection, wherein AAV9 and AAV9-NEM are administered by systemic delivery via tail-vein injection of WT Balb/c mice. N=2 mice/group.

FIG. 15 shows two graphs with the top providing a gLUC readout of primary murine BMSCs and osteoblasts 48 hours post transduction. MOI of 104 is the dose, N=3-5, and the bottom graph % of red or green BMSCs over osteoblast differentiation experiment. AAV9-NEM-mScarlet area significantly greater starting at Day5 post injection, MOI of 105.

FIG. 16 shows a graph representing the fold change of osteocalcin (Ocn) and AAV9-NEM-mScarlet positive bone surface relative to Ocn & AAV9-eGFP positive bone surface at 4 weeks and 8 weeks. N=3-4/group, Ratio paired T-Test used. AAV dose for the mice is 1.89×10¹¹ vg/kg.

### DETAILED DESCRIPTION

### Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes alleviation of the symptoms associated with a specific disorder or condition and/or preventing or eliminating said symptoms.

As used herein an “effective” amount or a “therapeutically effective amount” of a drug refers to a nontoxic but enough of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein the term “subject” without further designation is intended to encompass any warm blooded vertebrate domesticated animal (including for example, but not limited to livestock, horses, cats, dogs and other pets) and humans.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “vector” or “construct” designates a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “recombinant DNA” refers to an engineered ssDNA, such as a vector or plasmid.

The term “operably linked to” refers to the functional relationship of a nucleic acid with another nucleic acid sequence. Promoters, enhancers, transcriptional and translational stop sites, and other signal sequences are examples of nucleic acid sequences that can operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The term “tropism” as used herein in reference to viruses, defines the target cellular types that a virus preferentially, or has the capacity to, infect and establish a successful infection. Altered tropism refers to the resulting infecting capabilities of the modified AAV to infect one type of cell more or less relative to the unmodified AAV.

The term “chemical modification” as used herein defines a reaction where a protein is contacted with a reagent to produce a modified protein that comprises a chemical moiety covalent linked to the native protein.

### EMBODIMENTS

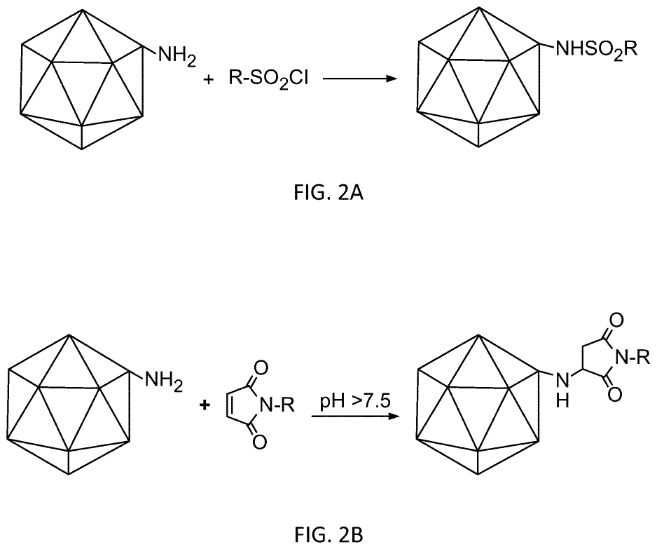

Disclosed herein are compositions and methods are provided for the delivery of biomolecules, molecular tools, or small molecules, to a subject's tissues. In accordance with the present disclosure, a modified adeno-associated virus (AAV) is provided including an AAV having a capsid formed from capsid proteins (VP1, VP2, and/or VP3) and a single stranded DNA (ssDNA), wherein a chemical moiety is covalently linked to the capsid to form the modified AAV, wherein the covalently linked chemical moiety has an altered tropism relative to an unmodified AAV. See FIG. 2A and FIG. 2B for cartoon representations of a modified AAV. In one embodiment, the ssDNA is a vector comprising recombinant DNA. When the AAV or modified AAV contains recombinant DNA, they may be referred to as rAAV or modified rAAV, respectively. Unless otherwise stated, a modified rAAV is included in the genus modified AAV.

The AAV employed to prepare the modified AAV may be of any serotype. In some embodiments, the AAV is a serotype selected from the group consisting of AAV2, AAV5, AAV8, and AAV9.

Additionally, the AAV may include an engineered capsid protein. For example, capsid AAV5× has one point mutation that resulted in a Met to Thr mutation, which is 30 Da less than wild-type AAV5 capsid VP3 sequence. It is well known in the art to generate an AAV or rAAV with an engineered capsid protein. In this manner, the capsid genes vp1, vp2, and/or vp3 are genetically modified resulting in an altered capsid proteins VP1, VP2, and/or VP3. The disclosure herein encompasses AAVs with engineered capsids, as well. In some embodiments, the AAV comprises an engineered capsid, which is then chemically modified as described herein. In some embodiments, the modified AAV comprises an AAV including an engineered capsid. In some embodiments, the modified rAAV comprises an AAV including an engineered capsid. In yet another embodiment the AAV to be modified is an rAAV, optionally wherein the rAAV is an rAAV selected from serotypes with engineered capsids. For the purposes of this disclosure capsid proteins and engineered capsid proteins conserve the at least one exposed lysine residue found in VP1, VP2, and/or VP3.

In some aspects, the chemical moiety contains a functional group capable of directly binding (covalently) or indirectly binding (such as via a linker) to at least one capsid protein. The functional group may bind to an amino acid that is naturally exposed and available for binding. In some embodiments, the amino acid is a lysine. The lysine may be found in the VP1, VP2, or VP3 capsid proteins. In some embodiments, the lysine is found in the VP3 capsid protein. The lysine may be found on the VP3 protein at position 326 or 259.

The functional group may comprise or consist essentially of a sulfonyl chloride or a maleimide.

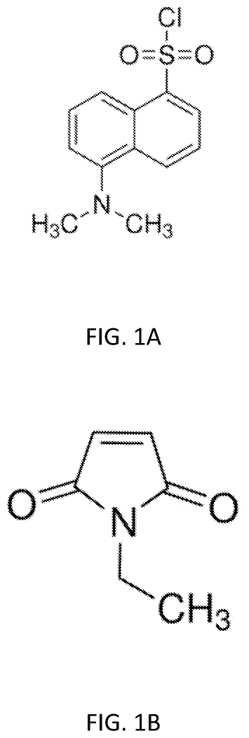

In some embodiments, the chemical moiety is selected from the group consisting of 5-(Dimethylamino) naphthalene-1-sulfonyl chloride (commonly known as Dansyl chloride, or in short “Dc” and having a CAS Registry Number 605-65-2), N-ethylmaleimide (in short “NEM” and having a CAS Registry Number 128-53-0) and 4-Nitrobenzenesulfonyl chloride (commonly known as Nosyl chloride, or in short “Nb” and having a CAS Registry Number 98-74-8). See FIG. 1A and FIG. 1B.

The modified AAV may include more than one bound chemical moiety. any of the preceding clauses, wherein the modified AAV comprises more than one chemical moiety. For instance, the more than one chemical moiety may be two or more, three or more, or even four or more. The more than one chemical moiety may be the same or a mixture of different chemical moieties. For example, a first chemical moiety may be generically defined as “A” and a second chemical moiety may be generically defined as “B.” The modified AAV may comprise one or more chemical moiety A, one or more chemical moiety B, or a mixture of A and B. In some instances, the modified AAV may contain a mixture of A, B, and a third chemical moiety generically defined as “C.”

Illustratively, the modified AAV may comprise one or more Dc, one or more NEM, one or more Nb, or a combination thereof. In some embodiments, the more than one chemical moiety is a Dc and either an NEM or an Nb. Alternatively, the more than one chemical moiety may be an NEM and either a Dc or an Nb. In yet another embodiment, the more than one chemical moiety is an Nb and either a Dc or an NEM.

When the AAV is modified as disclosed herein, the result is a change in tropism relative to the unmodified AAV. The following Examples and figures demonstrate this result. In general, an AAV may be modified with at least one chemical moiety to form a modified AAV so that the modified AAV gains the ability or preference for infecting a cell type or loses the ability or preference to infect a cell type. It is envisioned that delivery of biomolecules, small molecules, or molecular tools can be targeted to specific cell types and a reduction in broad bio-distribution.

As disclosed herein, chemically linked Dc, NEM, or Nb to the AAV capsids re-directs the tropism of AAV to specific tissues which can improve the therapeutic index of the AAV, significantly. Accordingly, one aspect of the present invention is the use of the modified AAVs disclosed herein as delivery vehicles for administering therapeutics to treat subjects suffering from various conditions and disease states.

In some embodiments, a modified AAV of one serotype may be administered to a subject to deliver a biomolecule (e.g., ssDNA, mRNA, peptide, etc.), small molecule, or molecular tool (e.g., fluorophore) to the subject's bone tissue. Additionally, a second modified AAV containing a different serotype, different chemical moiety, or both may be administered to the subject to deliver a biomolecule, small molecule, or molecular tool to the subject's muscle tissue.

Aspects of the modified AAV and methods of use are also set out in the following set of numbered clauses in which is described:
Clause 1. A modified adeno-associated virus (AAV) comprising (i) an outer shell comprising capsid proteins and at least one chemical moiety covalently bound to at least one capsid protein, the capsid proteins including VP1, VP2, and VP3, and (ii) a single strand of DNA (ssDNA).
Clause 2. The modified AAV of clause 1, wherein the ssDNA is recombinant DNA (rDNA).
Clause 3. The modified AAV of clause 1 or clause 2, wherein the at least one chemical moiety is covalently bound to a lysine residue present in the at least one capsid protein.
Clause 4. The modified AAV of any of the preceding clauses, wherein the chemical moiety is selected from the group consisting of 5-(Dimethylamino) naphthalene-1-sulfonyl chloride (“Dc”), 4-Nitrobenzenesulfonyl chloride (“Nb”), and N-ethylmaleimide (“NEM”).
Clause 5. The modified AAV of any of the preceding clauses 1-4, wherein the at least one chemical moiety comprises a sulfonyl chloride functional group.
Clause 6. The modified AAV of any of the preceding clauses 1-4, wherein the at least on chemical moiety comprises a maleimide functional group.
Clause 7. The modified AAV of any of the preceding clauses, wherein the modified AAV comprises a natural capsid or engineered capsids,
Clause 7.5 The modified AAV of any of the preceding clauses, wherein the modified AAV has a serotype selected from the group consisting of AAV2, AAV5, AAV5x, AAV8, and AAV9.
Clause 8. The modified AAV of any of the preceding clauses, wherein the modified AAV comprises more than one chemical moiety.
Clause 9. The modified AAV of clause 8, wherein the more than one chemical moiety is a Dc and either an NEM or an Nb.
Clause 10. The modified AAV of clause 8, wherein the more than one chemical moiety is an NEM and either a Dc or an Nb.
Clause 11. The modified AAV of clause 8, wherein the more than one chemical moiety is an Nb and either a Dc or an NEM
Clause 12. The modified AAV of any of the preceding clauses, wherein the chemical moiety is covalent bound to the VP1 protein
Clause 13. The modified AAV of any of the preceding clauses, wherein the chemical moiety is covalent bound to the VP2 protein.
Clause 14. The modified AAV of any of the preceding clauses, wherein the chemical moiety is covalent bound to the VP3 protein.
Clause 15. The modified AAV of any of the preceding clauses, wherein the chemical moiety is covalent bound to the VP1 protein, the VP2 protein, or the VP3 protein.
Clause 16. A method of altering the tropism of an adeno-associated virus (AAV) upon administration to a subject comprising: chemically modifying a capsid protein of the AAV by the covalent attachment of a chemical moiety to form a modified AAV, wherein the chemical moiety alters the tropism of the modified AAV compared to an unmodified AAV.
Clause 17. The method of clause 16, wherein the modified AAV includes recombinant DNA.
Clause 18. The method of clauses 16 or 17, wherein the chemical moiety is selected from the group consisting of 5-(Dimethylamino) naphthalene-1-sulfonyl chloride (Dc), or N-ethylmaleimide (NEM).
Clause 19. The method of clause 18, wherein the chemical moiety is 5-(Dimethylamino) naphthalene-1-sulfonyl chloride (Dc) and the altered tropism is an enhanced preferential uptake of the modified AAV, relative to the original unmodified AAV, by muscle tissue.
Clause 20. The method of clause 18, wherein the chemical moiety is N-ethylmaleimide (NEM) and the altered tropism is an enhanced preferential uptake of the modified AAV, relative to the original unmodified AAV, by bone tissue.
Clause 21. A modified recombinant adeno-associated virus (rAAV) comprising a capsid protein comprising a non-native chemical moiety covalently linked to the capsid protein, wherein the non-native chemical moiety alters the tropism of the modified rAAV relative to the original unmodified rAAV.
Clause 22. The modified rAAV of clause 21, wherein the modified rAAV is selected from serotypes AAV2, AAV5, AAV5x, AAV8, and AAV9.
Clause 23. The modified rAAV of clauses 21 or 22, wherein the chemical moiety is selected from the group consisting of 5-(Dimethylamino) naphthalene-1-sulfonyl chloride (Dc), and N-ethylmaleimide (NEM).
Clause 24. The modified rAAV of clause 22, wherein the chemical moiety is 5-(Dimethylamino) naphthalene-1-sulfonyl chloride (Dc) and the altered tropism is an enhanced preferential uptake of the modified rAAV, relative to the original unmodified rAAV, by muscle tissue.
Clause 25. The modified rAAV of clause 23 wherein the chemical moiety is N-ethylmaleimide (NEM) and the altered tropism is an enhanced preferential uptake of the modified rAAV, relative to the original unmodified AAV, by bone tissue.
Clause 26. A pharmaceutical composition comprising the modified AAV or modified rAAV of any one of the preceding clauses and a pharmaceutically acceptable carrier.
Clause 27. The modified AAV or modified rAAV of any one of the preceding clauses, wherein the altered tropism reduces the uptake of the modified AAV or modified rAAV by liver cells relative to the original unmodified AAV or unmodified rAAV.
Clause 28. The modified AAV or modified rAAV of clauses 20 and 25, or any other relevant clause, wherein the bone tissue is bone marrow.
Clause 29. The modified AAV or modified rAAV of any previous relevant clause for use as a therapeutic.
Clause 30. The modified AAV or modified rAAV of any previous clause or any relevant clause for use as a vehicle for delivery of a biomolecule, small molecule, or molecular tool with altered tropism.

### EXAMPLES

The following non-limiting examples are provided to further describe the modified AAVs and methods of making and using the modified AAVs.

### Example 1

Preparing and confirming modified AAV-Dc.

All recombinant adeno-associated viruses were produced by triple-transfection of HEK293 cells using three plasmids pRep-Cap, pVector, and pHelper-Ad. The capsid of each rAAV may be chosen among natural serotypes (for example AAV2, AAV8, or AAV9) and may also be synthesized by non-natural methods such as mutagenesis, capsid shuffling, and peptide insertions. rAAV were collected after 72-96 hr of transfection, and were purified by ion-exchange chromatography or iodixanol ultracentrifugation.

### Cell Lines

HEK293 cell line was purchased from ATCC for AAV vector production. The cells were cultured in DMEM supplemented with 10% fetal bovine serum, 100 μg/mL penicillin and 100 units/mL streptomycin (Invitrogen) and maintained in a humidified 37° C. incubator with 5% CO₂.

### Method of Capsid Chemical Modification:

Chemical reactions of the AAV capsid proteins with particular compounds (See FIG. 1A, FIG. 1B, FIG. 2A, and FIG. 2B) were performed to form covalent bonds and were performed in a buffered aqueous solution with an incubation time ranging from 1 to 24 hr. Incubation temperature may range from room temperature to 4° C. Chemical reactions were confirmed using SDS-PAGE and mass spectrometry.

### SDS-PAGE:

Mini-PROTEAN TGX stain-free pre-cast gel (7.5%, a polyacrylamide gel containing a proprietary tri-halo compound to make proteins fluorescent directly in the gel, Bio-Rad cat #456-8026) was used. A sample of 6 μL rAAV (~1E12 vg/mL) was added to 2 μL of 4× Laemmli sample buffer containing 10% of β-mercaptoethanol, and mixed well before being heated at 90° C. for 5 min. The sample was cooled to room temperature and loaded into the gel lanes, together with a standard marker. Running buffer was 1× Tris/Glycine/SDS, Bio-Rad cat #1610772). The assembly was set and connected. Voltage was set to be constant, at 200V. The gel was set to electrophorese for 30 min.

### Mass Spectrometry:

For intact LC-MS analyses: 1 μL of formic acid (10% FA) was added to 9 μL of AAVs (~2E10 vg) and incubated at room temperature for 10 minutes before injection.

For peptide mapping LC-MS/MS: 1E11 vg of AAVs was denatured in 6 M urea and 1 mM DTT at 90° C. for 20 min and alkylated with iodoacetamide (15 mM) for 30 min at room temperature in the dark. Reduced and alkylated samples were cooled to room temperature, diluted with 3 equivalent volumes of buffer (50 mM Tris-HCl, 1 mM CaCl₂), pH 7.5) to ensure the urea concentration down to ≤2 M. The samples were then digested by 0.4 μg of trypsin (PROMEGA) or chymotrypsin (THERMO) overnight at 37° C. Digested samples were cleaned up using peptide-cleanup C18 spin columns (AGILENT, Cat #5188-2750) right before LC-MS injection. A final 40 μL elution from each C18 cleanup column (followed the manufacture's protocol) in elution buffer (70% ACN, 0.1% TFA) was collected and used directly for LC-MSMS analysis. The purpose of this step is to remove interfering contaminants and increase peptide concentration.

Deconvolute Protein Software: Mass Hunter Bioconfirm B.08; Deconvolution algorithm=Maximum Entropy; USE mass range 50 k-90 k Da; M/z range: 700-3200 m/z; Adduct: proton.

Intact AAV Capsid LC-MS. The capsid of each AAV serotype consists of 3 VP proteins: VP1, VP2, and VP3, which share an overlapping open reading frame. VP1 is the largest with an approximate size of ~82 kDa, VP2 is around ~66 kDa, and VP3 is the smallest and most abundant with a size of ~60 kDa. To demonstrate the power of LC-MS analysis, a biological engineered capsid-AAV5x-resulted from a single point mutagenesis of VP3 position #VP3-M277T methionine to threonine, plus several chemical-engineered capsids AAV5x-Dc and AAV9-Dc were included in this study. One molecule of Dc resulting from a covalent bond formation with an amino group causes a 233.1 Da mass gain of that VP protein.

The exact masses of the VP3 proteins alone can distinguish different AAV serotypes. Analytical LC-MS and LC-MS/MS provide specificity, sensitivity, and speed in proteomics analysis, thus offer an ideal tool to distinguish AAV serotypes, and map engineered capsid proteins. Here, AAV9 was used for intact LC-MS related method development. Since AAV vectors are comprised of 60 monomers of VP proteins per capsid with VP1/2/3 in approximately 1:1:10 stoichiometric ratio, the capsid should be denatured into individual VP proteins before LC-MS injection to ensure proper protein ionization. Simple acidic condition of 0.1% formic acid (or 0.1% TFA) was added to the AAV samples 10 minutes before LC injection.

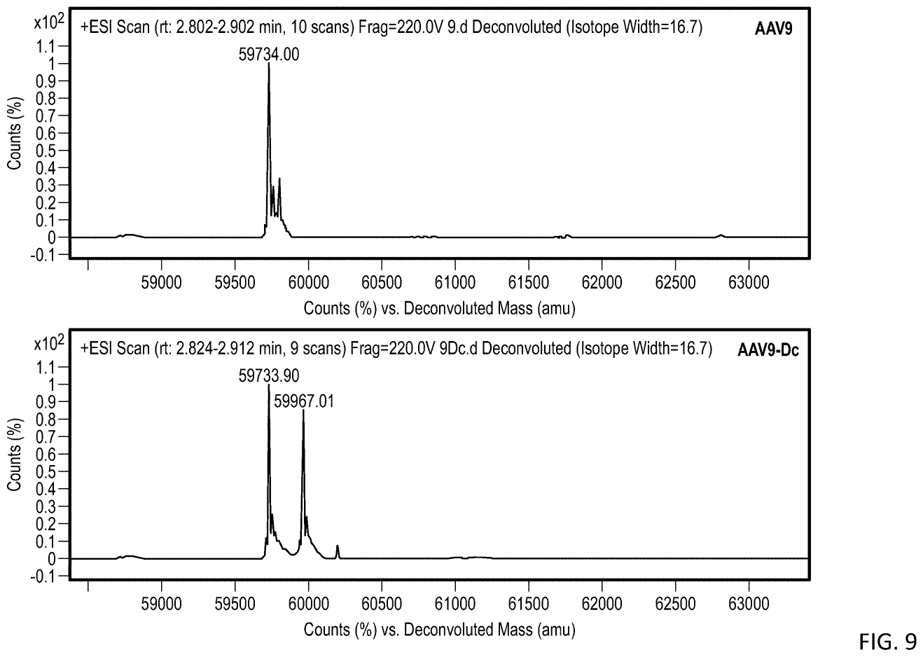

For fast and high-throughput purposes, a short column ZORBAX 300SB-C3 4.6×50 mm, 3.5 μm was used, as a single VP protein mass can sufficiently confirm an AAV capsid serotype variant (Table 1). The chromatographic profile of AAV9 is shown in FIG. 9. The capsid proteins fully elute at ~3.0 min in each intact LC-MS run, indicating that each run can end at 4 min and still provide a high-throughput value for multiple batch screening and characterization of AAV serotypes. Wild-type AAV9's raw and deconvoluted mass spectra are also shown in FIG. 8 (top panel). For comparison, chemically engineered AAV9-Dc spectra are shown in FIG. 8 (bottom panel). The exact masses of each VP protein were detected as shown by the deconvoluted mass spectra, with VP3 (59,734 Da) displaying the most intense signal, reflecting its relative abundance compared to the other VP proteins, VP2 (66,211 Da) and VP1 (81,292 Da). We calculated the stoichiometric ratio of these VP1/2/3 to be 7/9/100 (FIG. 8). From the AAV9-Dc deconvoluted mass spectrum, an additional mass gain of 233.1 Da was found as the second most intense peak right next to the VP3 peak. For better data interpretation, FIG. 9 shows a close-up look around VP3 peaks of AAV9 and AAV9-Dc.

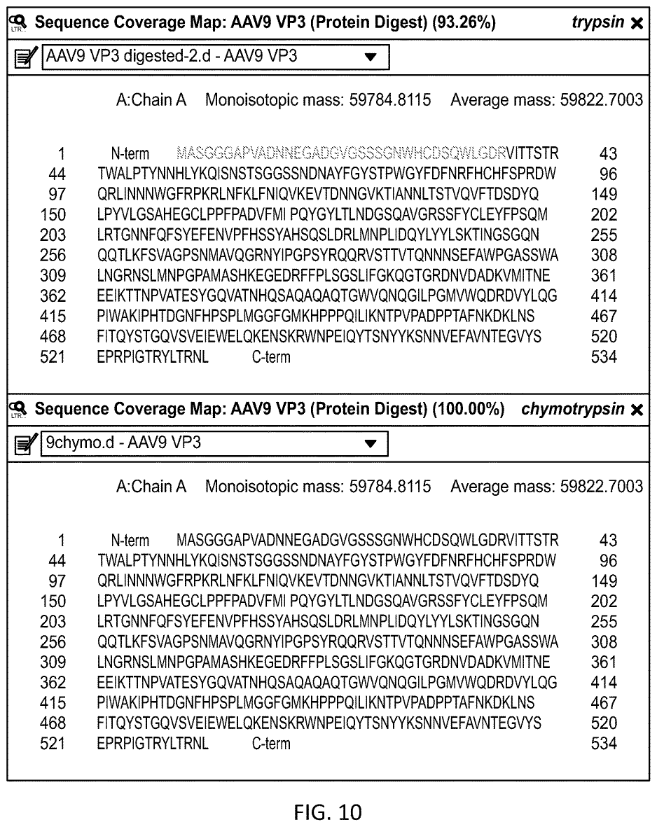

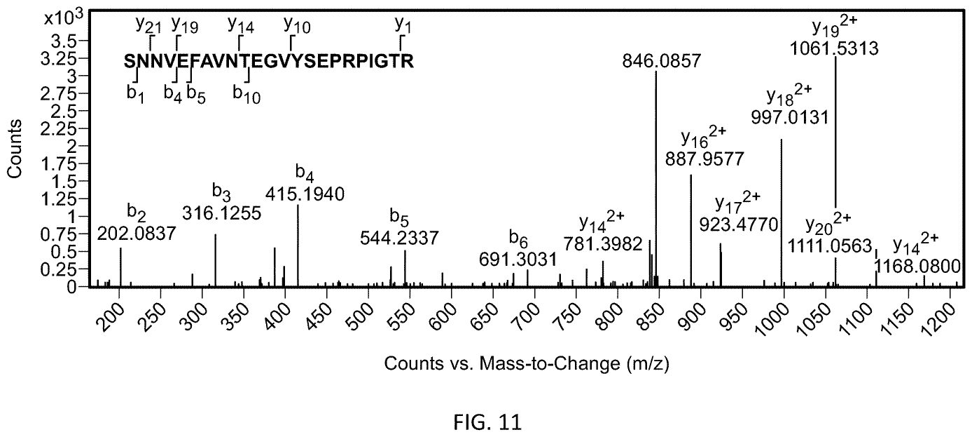

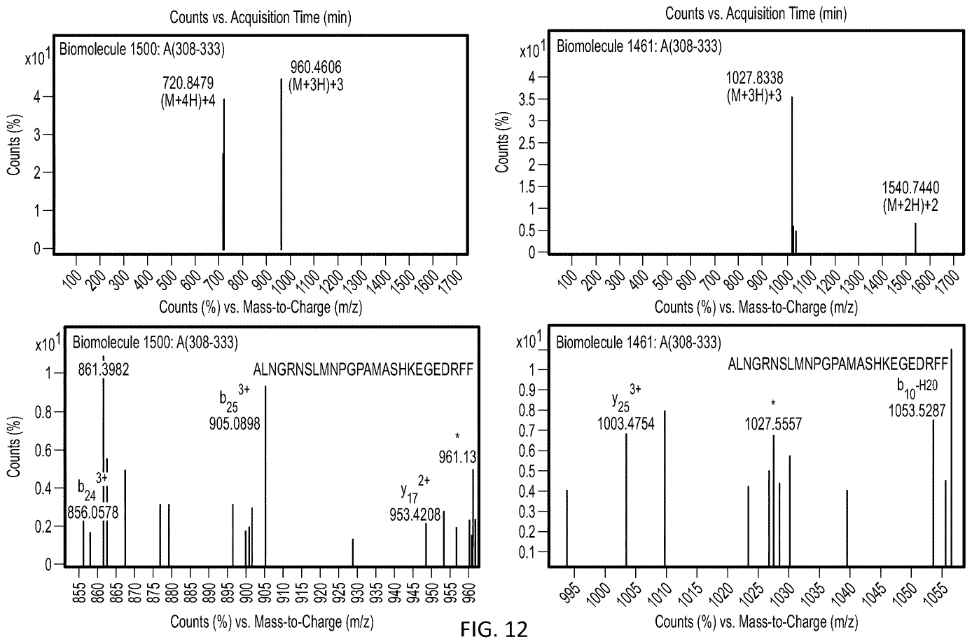

Unlike Western blots, where small-molecule chemical modifications can only be confirmed indirectly through the attachment of a reporter or epitope tag to the molecule being tested, LC-MS directly measured and confirmed the covalent chemical addition in just a few minutes. For LC-MS/MS method development, trypsin and chymotrypsin proteases were used to digest AAVs. Trypsin cleaves peptides on the C-terminus of Lys and Arg, while chymotrypsin cleaves at the carboxyl side of aromatic amino acids Tyr, Phe, and Trp. With limited Lys and Arg residues, trypsin gave less sequence coverage than chymotrypsin. Furthermore, with chemical modifications targeting Lys, cleavages by trypsin might be affected, and therefore chymotrypsin was superior in this case. Specifically, AAV9 VP3 protein was peptide mapped and had a sequence coverage of 93% (trypsin digested) and 100% (chymotrypsin digested) as shown in FIG. 10. Even though VP2 and VP1 are lower in abundance compared to VP3 monomers, their sequence coverage was found to be 94% for VP2 and 93% for VP1 of AAV9. An example of a peptide MSMS spectrum with the b and y ions is shown in FIG. 11. These m/z values of fragmented ions are unique fingerprint patterns for accurate peptide characterization.

Since chymotrypsin provided higher sequence coverage of AAV9 capsid proteins compared to trypsin, it was used to map the engineered capsid AAV9-Dc. As shown in FIG. 12, site-specific position of amino acid Lys at VP3#326 was found to be conjugated with one Dc molecule, hence the mass gain of 233.1 Da, while the corresponding wild-type sequence did not show the mass gain. This position was confirmed to be an exposed Lys on the intact capsid of AAV9 using the 3D-structure (3ux1) from the Protein data bank.

Additionally, peptide mapping analyses were performed for AAV8, AAV5x, and AAV5x-Dc to assess the method's general applicability across AAV serotypes. These capsids gave a sequence coverage of at least 92% (AAV5x VP3) and the highest of 100% (AAV8 VP3 and VP2). The most accessible amino acid regarding the Dc-molecule covalent modification was found to be Lys-VP3#326 of AAV9 (FIG. 12) and Lys VP3 #-259.

TABLE 1
Mass Accuracy of AAV Capsid Intact LC-MS

| | AAV Capsid Proteins (Da) | | | | | | | | |
|---|---|---|---|---|---|---|---|---|---|
| | VP1 | | | VP2 | | | VP3 | | |
| | | | Mass | | | Mass | | | Mass |
| AAV | | | Accuracy | | | Accuracy | | | Accuracy |
| serotype | Theoretical | Observed | ppm | Theoretical | Observed | ppm | Theoretical | Observed | ppm |
| AAV2 | 81,856 | 81,855 | 12.22 | 66,488 | 66,490 | 30.08 | 59,974 | 59,975 | 16.67 |
| AAV5x | 80,306 | 80,309 | 37.36 | 65,253 | 65,255 | 30.65 | 59,433 | 59,434 | 16.83 |
| AAV8 | 81,667 | 81,668 | 12.24 | 66,519 | 66,519 | 0.00 | 59,805 | 59,807 | 33.44 |
| AAV9 | 81,291 | 81,292 | 12.30 | 66,210 | 66,211 | 15.10 | 59,733 | 59,734 | 16.74 |

### Preparing and Confirming Modified AAV-NEM

Purified rAAVs (AAV8 and AAV9) were reacted with NEM, Biotin-maleimide, or Rhodamine-maleimide (SIGMA) in PBS buffer at pH 8, with a molar ratio of 10,000 molecules/vg, overnight at 4 C. The reacted samples were then filtered, and buffer exchanged extensively (~10 times) with PBS buffer pH 7.2 using Vivaspin20 100K MWCO and concentrated down to around the initial volume of the rAAV. The chemically modified rAAVs were then titrated alongside with the un-modified rAAVs counterparts for titer determination before any downstream analyses and animal injections.

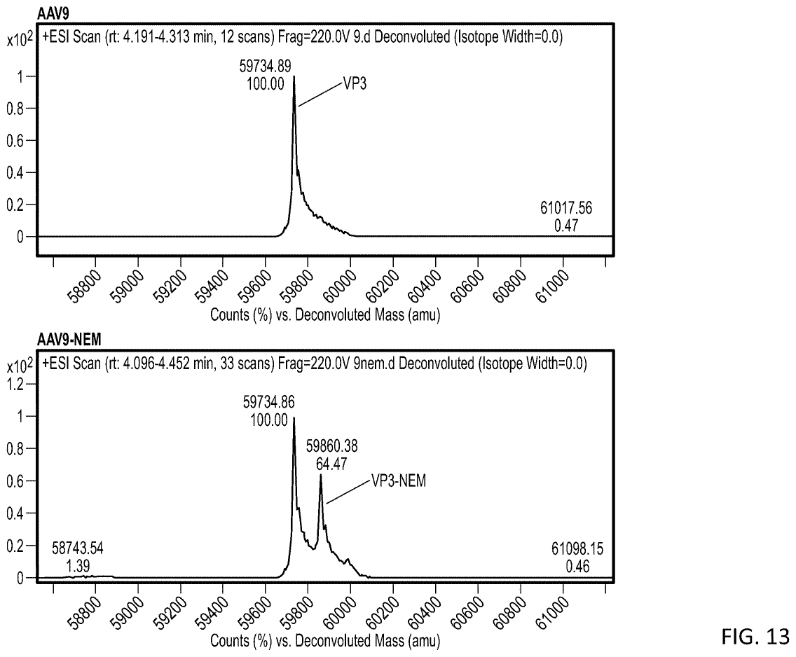

LC-MS, as described above, was used to confirm the production of the modified AAV-NEM. Turning to FIG. 13 (top panel), the deconvoluted mass spectrum of AAV9 VP3 shows the exact mass detected (59,734 Da), which almost exactly matches its theoretical mass (59,733 Da). FIG. 13 (bottom panel) shows the same spectrum for AAV9-NEM VP3, and the exact mass gain of one NEM molecule (~125 Da) was detected to be 59,860 Da, which confirmed the modification of NEM on AAV9. General morphology of AAV9-NEM compared to its unmodified counterpart AAV9 by transmission electron microscopy (TEM), revealed similar morphology among the two variants (images not shown).

### Example 2

### In Vitro Testing of the Modified AAVs

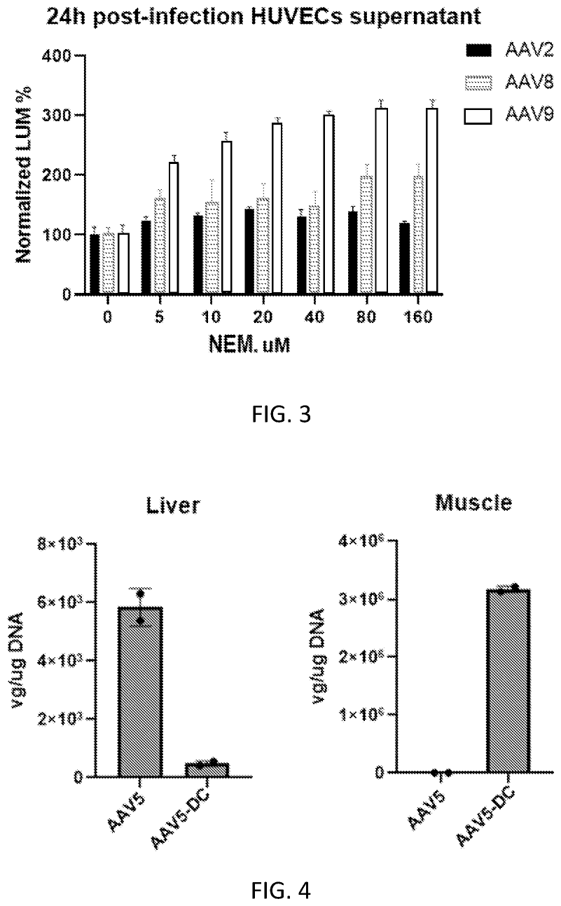

Turning to FIG. 3, to assess the phenotypic change of AAV-NEM, in vitro transduction assays were performed by infecting the same multiplicity of infection (MOIs) of AAV-NEM and their unmodified counterparts on different cell lines (GM16095, HeLa, and HUVEC). All of the vectors tested (AAV2, AAV8, and AAV9) packaged (pAAV-CB-gLUC) as indicated by the outcome measurements of Gaussia luciferase. No significant difference in transduction was measured on human fibroblast cells GM16095 and HeLa cells, respectively. However, the transduction of AAV9-NEM on human umbilical vein endothelial cells (HUVEC) was significantly higher than the WT AAV9 (FIG. 3). Additionally, no morphological differences between unmodified and NEM-modified AAV9 were observed.

### BMSC Differentiation

Primary bone marrow stromal cells from 8-10-week-old male Balb/C mice were collected and cultured. Briefly, mouse hindlimbs were extracted, stripped of fascia and tissue, and the proximal epiphyses were cut off. Bones were inserted into 0.5 mL tube punctured at the bottom that was placed into 1.5 mL tube containing sterile 1×PBS. Bones were centrifuged, and the resulting pellets were grown in 10% α-MEM in 24 well plates. For wells selected for osteogenic differentiation, osteogenic medium (10% α-MEM containing 50 μg/mL ascorbic acid [SIGMA, #A-5950] and 5 mM beta-glycerophosphate [SIGMA, #G9891-25G]), was used for up to 20 days.

### In Vitro Cell Line Experiments

For Luciferase in vitro experiments, rAAV9 or rAAV9 modified with NEM driving expression of Guassia Luciferase (gLUC) was transduced into cells at MOI of 10\hat{ }4. For experiments with BMSCs, luciferase readout was conducted 48 hrs post transduction on days 3, 5, 8, 14, and 21 of culture. Osteogenic media was started on day 6. Cell media was collected, and 20 uLs were placed into wells of an all-white, 96-well plate. 50 uLs of 1× coelenterazine was added using a multichannel pipet, and plate was read immediately for luminescence after a brief shake at an integration time of 10 ms.

For eGFP and mScarlet in vitro experiments, primary BMSCs were transduced with rAAV9-eGFP or rAAV9-NEM-mScarlet at MOI of 10\hat{ }5 after reaching confluency at D14 of culture. After AAV transduction, certain BMSCs were cultured in osteogenic media for the remainder of the study. Fluorescent images were taken at Days 2, 5, 8, and 14 post-transduction. 3-4 images per group were analyzed for % green or red positive area via Metamorph Software. Calcified nodules were noticeable 48 hours after starting the osteogenic media cultures.

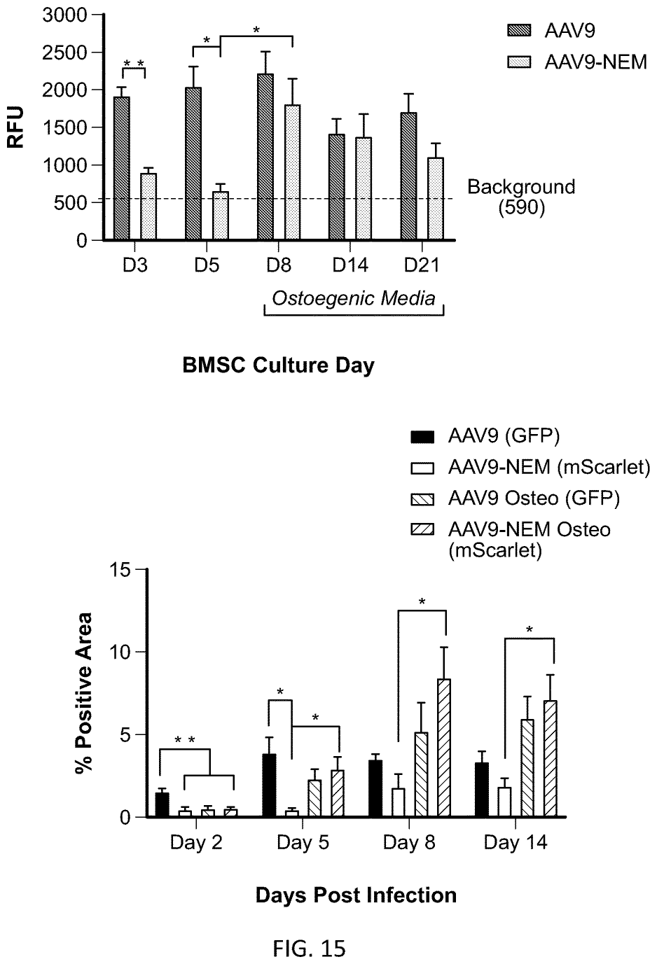

To investigate these potential transduction differences between AAV9 and AAV9-NEM in a more controlled environment, we isolated primary mouse bone marrow stromal cells (BMSCs) from bone marrow of uninjected Balb/C male adult mice for two sets of studies. We employed AAV9 or AAV9-NEM driving expression of gLUC under control of the CB promoter for the first set of experiments, and AAV9-CB-eGFP along with AAV9-NEM-CB-mScarlet for the second set. At Days 3 and 5 of the gLUC experiments, BMSCs transduced with AAV9 (MOI of 10\hat{ }4) exhibited significantly higher luciferase readout compared to AAV9-NEM. Interestingly, BMSCs transduced on Day 8 (48 hours in osteogenic medium) with either AAV9 or AAV9-NEM resulted in similar luciferase readings; This trend continued to be seen on Days 14 and 21 (FIG. 15). This data suggests that the NEM modification of AAV9 alters its transduction in vitro.

For the second set of experiments, our goal was to elucidate more details about this observed difference in AAV9-NEM transduction in undifferentiated BMSCs compared to those pushed towards an osteoblast lineage. BMSCs were grouped based on AAV9 or AAV9-NEM transduction as well as growth in osteogenic medium or regular α-MEM medium after 7 days of initial growth in α-MEM. As we observed in the gLUC studies, AAV9-eGFP transduced the greatest percentage of BMSCs in α-MEM at Days 2 and 5 compared to AAV9-NEM-mScarlet in α-MEM or either of the osteogenic culture systems. On Day 5, however, the AAV9-NEM-mScarlet signal in the osteogenic culture was significantly greater than that seen in the AAV9-NEM-mScarlet α-MEM culture. This significant difference in red cells persisted on Days 8 and 14 of this experiment (FIG. 15). In addition, the percentage of cells transduced with AAV9-eGFP steadily increased in osteogenic cultures, matching that of the α-MEM cultures. Upon further analysis of both osteogenic cultures, a majority of transduced cells localized to the calcified nodules at Days 8 and 14. Overall, the results from these studies suggest that the NEM modification on AAV9 can alter its transduction tropism in vitro in BMSC cultures, and that AAV9-NEM transduces osteoblastic cells more successfully than undifferentiated BMSCs in culture settings.

The chemically modified AAV9-NEM transduced HUVECs and murine BMSCs differentiated toward osteoblasts greater than WT AAV9 in culture. In the osteoblast cultures, a majority of the transduced cells localized to calcified nodules, indicative of osteoblastic cells. Moreover, NEM modification of AAV9 altered in vitro transduction of primary BMSCs and those differentiated to osteoblasts (FIG. 15).

FIG. 15 shows NEM modification Alters the Transduction of AAV9 in BMSCs Differentiated to Osteoblasts In Vitro. gLUC readout of primary murine BMSCs and osteoblasts 48 hours post transduction. MOI of 10\hat{ }⁴ is the dose. N=3-5, and % of red or green BMSCs over osteoblast differentiation experiment. AAV9-NEM-mScarlet area significantly greater starting at Day5 post injection, MOI of 10\hat{ }5.

### Example 3

### In Vivo Testing of the Modified AAVs.

Mouse Studies with AAV9: Each specific experiment was designed so that each vector (AAV9 or AAV9-NEM) packaged a unique transgene of interest so that they allowed for different purpose of analyses. For the bio-distribution study, AAV9 and AAV9-NEM each carried a unique barcode for retrospective analysis of the viral genome which determined the capsid tropism. DNA extractions from mouse tissues were performed using the Qiagen DNeasy Blood & Tissue Kit per manufacturer's protocol. For the AAV9-eGFP/AAV9-NEM-mScarlet in vivo study, 8-10 week old male Balb/C mice were intravenously injected via the tail vein with 5×10¹⁰ vg (viral genomes) with AAV9/AAV9-NEM concoction. This dose approximately equates to 1.89×10¹¹ vg/kg based on the average weight of 26.5 g for the mice at injection used in these studies. PBS was injected into control animals. Prior to injection, mice were weighed and placed under a heat lamp for 10 mins to dilate vessels to ease injections and then monitored for 30 mins post injection for any complications. At sacrifice, we measured a final body weight as well as liver and spleen weights before euthanasia and soft tissue collection with no abnormal observation.

Tissue Processing, Microscopy, & Immunofluorescence. For AAV9/AAV9-NEM in vivo studies, liver, spleen, heart, lungs, and hindlimbs were collected from mice and fixed in 4% PFA for 48 hrs at 4 C, then transferred to 20% sucrose for 24 hrs, and then to 25% sucrose at 4 C. Livers and hindlimbs were embedded in OCT or a 1:1 ratio of OCT and 25% sucrose. Tissues were imaged on a Zeiss Axio Observer 7 microscope at 10× and 20× objectives. Immunofluorescence was performed on sequential liver and hindlimb sections for mouse Albumin (Proteintech, 16475-1-AP, 1:500 dilution), F4/80 (BioxCell, BE0206, 1:200 dilution), and Endomucin (EMCN, sc-53940, 1:100). A donkey anti-rat or anti-rabbit secondary antibody Alexa-fluor 647 (Invitrogen A78947 & A32795, 1:500) was applied prior to imaging. For Osteocalcin (Ocn, Proteintech #23418-1-AP, 1:150) staining, we performed a similar protocol; a donkey anti-rabbit secondary antibody Alexa 647 (Invitrogen A32795, 1:500) was used before imaging. For immunofluorescence analyses, “% Positive Area” was calculated using color thresholds for red (mScarlet), green (eGFP), yellow (mScarlet & eGFP), or purple (Alexa 647). The entire image (Area=6.1E\hat{ }6 pixels) was analyzed for a specific color area to get the % Positive area. 2-3 images per tissue were analyzed and averaged for the final graphs.

Capsid-modified rAAV vectors were prepared for in vivo studies using the same triple-transfection method and purified either by iodixanol ultracentrifugation or by ion-exchange chromatography (used SP-HP column and Q-HP column of AKTA system). The same titer of unmodified and modified capsid (about 5-10E10 vg per serotype with each serotype's genome has a unique DNA barcode or different transgene) was IV tail-vein injected into each Balb/c or BLK6 mouse. At week 4 post-injection, the mice were sacrificed, and their organs were harvested for gene expression analyses or DNA extraction using Qiagen DNeasy Blood & Tissue Kit. qPCR analyses were performed using DNA-barcode primers and hydrolysis probes for specific serotypes of AAV to quantify the vector genomes.

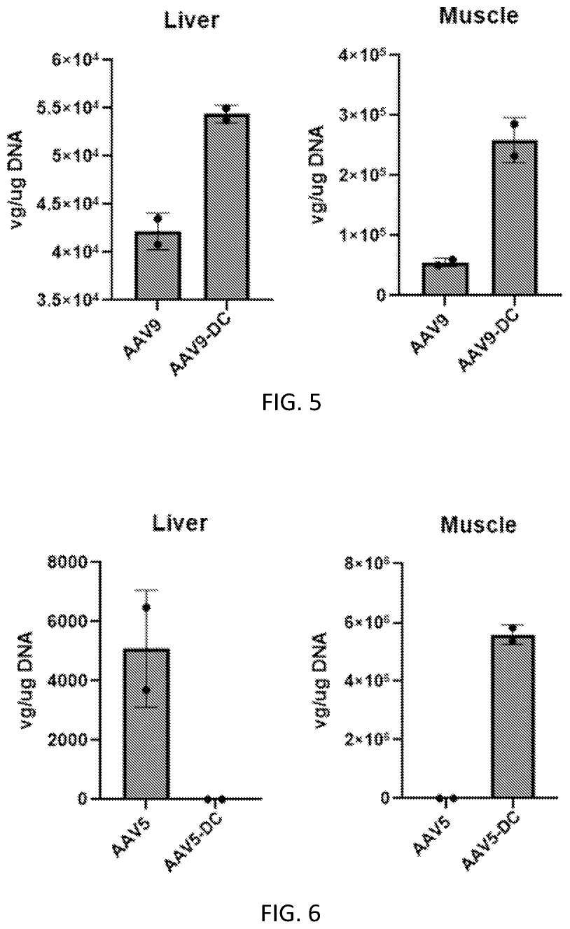

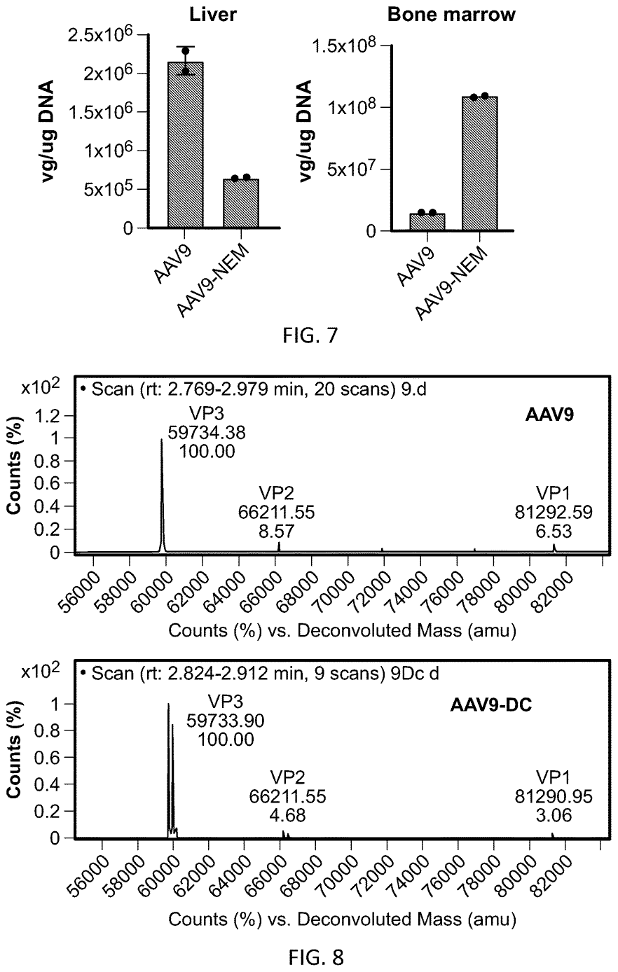

The in vivo data presented in FIGS. 4 through 7 were performed on mice using these modified-capsid rAAV vectors with multiple replicates. They showed significant improvement of these modified vectors compared to unmodified capsid AAV to specific tissues.

### In Vivo Evaluation of AAV9-NEM's Transduction and Tropism

Biodistribution analyses of AAV9-NEM compared to unmodified AAV9 were further evaluated by systemic delivery in WT Balb/c male mice. The study used a dose of AAV9 and AAV9-NEM (5×10\hat{ }10 vg per AAV; each capsid package a unique barcode for retrospective analysis) was injected IV. At week 1 or week 4 post injection, the mice were sacrificed for tissue collection and analyses.

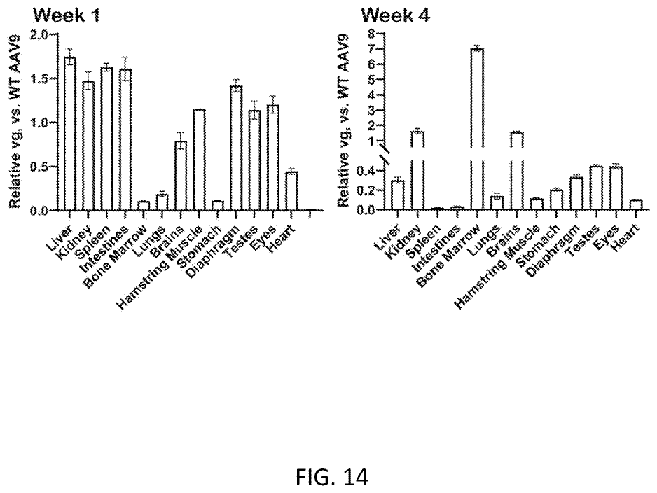

As shown in FIG. 14, DNA analysis of tissues at week 1 show the early changes in tropism of AAV9-NEM vs. AAV9, as that AAV9-NEM moderately increased vector DNA in some tissues (e.g., liver, kidney, or spleen) while decreased in others (e.g., bone marrow, lungs, or stomach). At week-4 post-injection, the change in tropism of AAV9-NEM was found to be more enhanced in the bone marrow, where vector genome (vg) DNA was around 7-fold higher than the unmodified AAV9 (FIG. 14). To examine the gene expression (gLUC), in vivo bioluminescence imaging was used with IP injection of the substrate coelenterazine with a dose of 200 mL/mouse (30 mg/mouse) and performed immediately after the substrate injection. AAV9-NEM gave a brighter signal (double the light intensity emitted) compared to AAV9 at week-4 post injection. These results indicate that the chemical-engineered capsid AAV9-NEM indeed changed the tropism and also enhanced the transduction of wild-type capsid AAV9.

The location of the mScarlet-positive AAV9-NEM cells in the bone tissues of the mice, and the presence of Cd90 and Cxcr4-positive AAV9-NEM bone marrow suggest that cells involved in bone turnover could be a target of this modified AAV9 vector.

### AAV9-NEM-mScarlet and Osteocalcin Positive Cells Observed at the Marrow/Calcified Bone Interface.

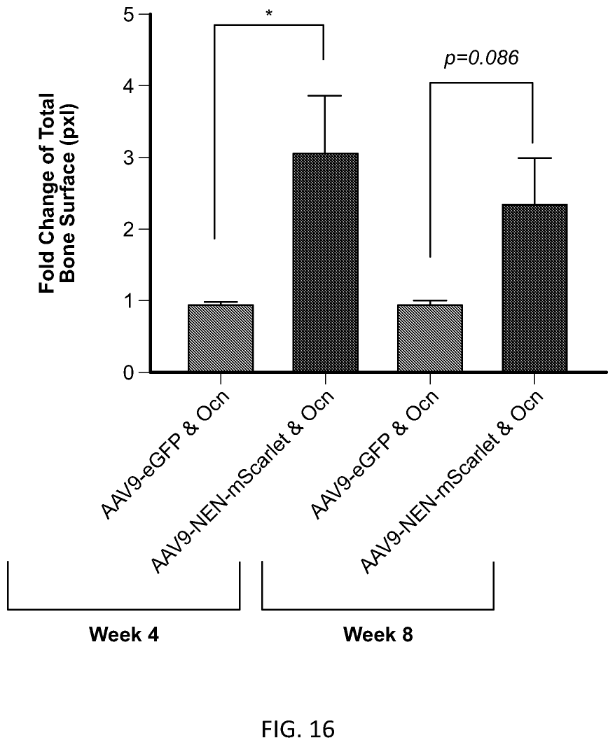

Based on these in vitro data described in Example 2 and the location of the mScarlet positive cells in the bone sections, we chose to further analyze the in vivo study to assess co-localization of the osteoblast marker, osteocalcin (Ocn), with AAV9-eGFP and AAV9-NEM-mScarlet transduced cells in the liver and bone. Ocn, a secreted factor produced by mature osteoblasts that has various endocrine effects both in the bone and in other organs, was detected throughout the bone, in both the marrow as well as at the interface lining the calcified tissue at 4 weeks and 8 weeks post injection; no osteocalcin was observed in the livers (data not shown). Interestingly, co-localization between mScarlet-positive cells along the edge of the calcified bone and Ocn (purple) was consistently observed in the bone sections, suggestive of osteoblasts. Regarding eGFP positive areas, some Ocn co-localization is seen in the marrow area, but minimal eGFP/Ocn co-localization was observed along the edge of the calcified bone (FIG. 16).

In a murine in vivo biodistribution study using a dose of 5E\hat{ }¹⁰, vector genomes delivered by AAV9-NEM were detected at higher concentrations in bone marrow and at lower concentrations in the liver 4 weeks post injection compared to WT AAV9 (. This result is indicative of the change in tropism by AAV9-NEM as the viral genomes delivered to the bone marrow were ~7-fold higher than the unmodified counterpart AAV9. In mice injected with the dual vector concoction (both the NEM-modified and WT vectors), AAV9-NEM and AAV9 transduced separate populations of cells in both liver and bone marrow as each single positive population in both organs was significantly greater than double-positive cells up to 8 weeks post injection.

In bone marrow isolates from mice, a greater percentage of AAV9-NEM transduced cells compared to AAV9 transduced cells expressed Cd31, Cd34, or Cd90. We detected elevated co-localization of Albumin-positive hepatocytes compared to minimal co-localization of F4/80+ Kupffer cells/macrophages or Emcn+ endothelial cells with AAV-transduced liver tissue, while in the bone marrow, AAV9-NEM exhibited co-localization with Emen and osteocalcin (Ocn) positive populations (Not shown). Many of the Ocn/AAV9-NEM double positive cells were located at the interface between marrow and calcified bone tissue (FIG. 16).

1. A modified adeno-associated virus (AAV) comprising:
(i) an outer shell comprising capsid proteins and at least one chemical moiety covalently bound to at least one capsid protein, the at least one capsid protein selected from VP1, VP2, or VP3, and
(ii) a single strand of DNA (ssDNA), wherein the ssDNA is recombinant DNA (rDNA), wherein the at least one chemical moiety is covalently bound to a lysine residue present in the at least one capsid protein, and
wherein the at least on chemical moiety alters the tropism of the modified AAV relative to an unmodified AAV.

2. The modified AAV of claim 1, wherein the chemical moiety includes a functional group selected from the group consisting of a maleimide and a sulfonyl chloride.

3. The modified AAV of claim 2 wherein the chemical moiety is selected from the group consisting of 5-(Dimethylamino) naphthalene-1-sulfonyl chloride (“Dc”), 4-Nitrobenzenesulfonyl chloride (“Nb”), and N-ethylmaleimide (“NEM”).

4. The modified AAV of claim 1, wherein the modified AAV includes an engineered capsid protein.

5. The modified AAV of claim 1, wherein the modified AAV comprises two or more chemical moieties.

6. The modified AAV of claim 5, wherein the two or more chemical moieties each have the same functional group.

7. The modified AAV of claim 5, wherein the two or more chemical moieties each have a different functional group.

8. The modified AAV of claim 5, wherein the two more chemical moieties include a Dc and either an NEM or an Nb.

9. The modified AAV of claim 5, wherein the two or more chemical moieties include an NEM and either a Dc or an Nb.

10. The modified AAV of claim 5, wherein the two or more chemical moieties include an Nb and either a Dc or an NEM.

11. The modified AAV of claim 1, wherein the at least one chemical moiety is covalent bound to the VP1 protein.

12. The modified AAV of claim 1, wherein the at least one chemical moiety is covalent bound to the VP2 protein.

13. The modified AAV of claim 1, wherein the at least one chemical moiety is covalent bound to the VP3 protein.

14. The modified AAV of claim 1, wherein the at least one chemical moiety is covalent bound to the VP1 protein, the VP2 protein, or the VP3 protein.

15. A method of altering the tropism of an adeno-associated virus (AAV) upon administration to a subject comprising:
chemically modifying a capsid protein of the AAV by the covalent attachment of a chemical moiety to form a modified AAV, wherein the chemical moiety alters the tropism of the modified AAV compared to an unmodified AAV.

16. The method of claim 15, wherein the modified AAV includes recombinant DNA.

17. The method of claim 15, wherein the chemical moiety is selected from the group consisting of 5-(Dimethylamino) naphthalene-1-sulfonyl chloride (Dc), or N-ethylmaleimide (NEM).

18. The method of claim 17, wherein the chemical moiety is 5-(Dimethylamino) naphthalene-1-sulfonyl chloride (Dc) and the altered tropism is an enhanced preferential uptake of the modified AAV, relative to the original unmodified AAV, by muscle tissue.

19. The method of claim 17, wherein the chemical moiety is N-ethylmaleimide (NEM) and the altered tropism is an enhanced preferential uptake of the modified AAV, relative to the original unmodified AAV, by bone tissue.

20. The method of claim 15, wherein the altered tropism reduces the uptake of the modified AAV by liver cells relative to the original unmodified AAV.
